(19) United States
(12) Patent Application Publication
(10) Pub. No.: US 2023/0355959 A1
(43) Pub. Date: Nov. 9, 2023

(54) SHEET MASK DEVICE

(71) Applicant: REZIENA INC., Gangnam-gu, Seoul (KR)

(72) Inventors: Tae Kyun KIM, Goyang-si, Gyeonggi-do (KR); Seung Woo SHIN, Goyang-si, Gyeonggi-do (KR)

(21) Appl. No.: 18/248,782

(22) PCT Filed: Nov. 12, 2020
(86) PCT No.: PCT/KR2020/015911

§ 371 Date: Apr. 12, 2023

(30) Foreign Application Priority Data
Oct. 13, 2020 (KR) 10-2020-0131534

(51) Int. Cl. A61N 1/30; A61N 1/04

(52) U.S. Cl. CPC A61N 1/0428; A61N 1/0492; A61N 1/303

(57) ABSTRACT

A sheet mask device includes a base sheet having a shape to cover the skin; an electrode unit formed on the base sheet to allow a current for iontophoresis to flow; and a power supply unit formed on the base sheet and connected to the electrode unit to supply electric energy. The power supply unit includes a battery activated by the liquid composition supplied to the base sheet; and a display unit configured to visually display whether or not the battery is activated.

### TECHNICAL FIELD

The present disclosure relates to a sheet mask device.

### BACKGROUND

In general, a sheet mask is a face-shaped fabric sheet that is soaked in nutrient and moisture components and helps recover skin conditions. If you place the sheet mask on the skin and remove it after a certain amount of time has passed, a thin layer is formed on the skin surface and inhibits evaporation of moisture and supplies nutrients. Thus, the sheet mask improves skin conditions within a short time.

In this regard, Korean Patent No. 2012929, which is one of prior arts, relates to a cosmetic mask pack that includes a non-woven layer containing a cosmetic composition, a binder layer coated on a surface of the non-woven layer and an aluminum thin film layer attached to the non-woven layer by the binder layer with an aluminum pattern attached thereto and configured to sense a facial skin temperature and change in color depending on the skin temperature, and, thus, the cosmetic mask pack maintains the skin temperature and allows the skin to breathe.

However, conventional technologies including general sheet masks can form a thin layer on the skin surface, but have limitations in supplying nutrients deep into the skin. Therefore, in order to supply nutrients deep into the skin, expensive devices or therapies such as an LED mask, a galvanic massage device and iontophoresis therapy are needed in addition to the use of a sheet mask.

### DISCLOSURE OF THE INVENTION

### Problems to Be Solved by the Invention

The present disclosure is to solve the above-described problem, and provides a sheet mask device that has the effect of supplying nutrients deep into the skin just with the attachment of a sheet mask like an iontophoresis or galvanic device.

The problems to be solved by the present disclosure are not limited to the above-described problems. There may be other problems to be solved by the present disclosure. means for solving the problems.

According to an aspect of the present disclosure, a sheet mask device includes a base sheet having a shape to cover the skin; an electrode unit formed on the base sheet to allow a current for iontophoresis to flow; and a power supply unit formed on the base sheet and connected to the electrode unit to supply electric energy. The power supply unit includes a battery activated by the liquid composition supplied to the base sheet; and a display unit configured to visually display whether or not the battery is activated.

The display unit includes an LED element that is turned on by electric energy supplied from the battery.

The LED element changes in at least one of a flicker pattern, brightness or color depending on an intensity of the electric energy supplied from the battery.

The LED element is turned on for a predetermined amount of time from when the battery is activated.

The sheet mask device further includes a case that accommodates the base sheet and the liquid composition to be isolated from each other, and deisolates the base sheet and the liquid composition when a user applies a pressure to the case.

The battery includes an anode configured to receive the liquid composition; a cathode configured to receive an oxygen from an air; and an electrolyte layer interposed between the anode and the cathode.

The electrode unit includes a first electrode pattern formed on a part of the base sheet and connected to an electrode of the battery; and a second electrode pattern formed on another part of the base sheet and spaced apart from the first electrode pattern, and connected to the other electrode of the battery.

The electrode unit is formed of a material containing silver (Ag) by sputtering on the base sheet.

The base sheet includes a first sheet that is a fibrous sheet to be in contact with the skin; and a second sheet that is formed of a metal-containing material on the first sheet and is coupled to the electrode unit.

The above-described aspects are provided by way of illustration only and should not be construed as liming the present disclosure. Besides the above-described embodiments, there may be additional embodiments described in the accompanying drawings and the detailed description.

### Effects of the Invention

According to any one of the above-described means for solving the problems of the present disclosure, it is possible to provide a sheet mask device that generates a microcurrent with a cosmetic composition and deliver the cosmetic composition deep into the skin.

Also, it is possible to provide a sheet mask device that can exercise iontophoresis or galvanic functions on the skin just by attaching a sheet mask onto the skin.

The present disclosure is to solve the above-described problem, and provides a sheet mask device that has the effect of supplying nutrients deep into the skin just with the attachment of a sheet mask like a galvanic device.

### BRIEF DESCRIPTION OF THE DRAWINGS

FIG. 1 illustrates a sheet mask device according to the present disclosure.

FIG. 2 is a conceptual diagram illustrating a power supply unit shown in FIG. 1.

FIG. 3 is a conceptual diagram illustrating a battery shown in FIG. 2.

FIG. 4 shows an example operation of a display unit shown in FIG. 2.

FIG. 5 is a cross-sectional view of a base sheet shown in FIG. 1.

### BEST MODE FOR CARRYING OUT THE INVENTION

Hereinafter, embodiments of the present disclosure will be described in detail with reference to the accompanying drawings so that the present disclosure may be readily implemented by a person with ordinary skill in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element. the term “comprises or includes” and/or “comprising or including” used in the document means that one or more other components, steps, operation and/or existence or addition of elements are not excluded in addition to the described components, steps, operation and/or elements unless context dictates otherwise.

Hereinafter, an exemplary embodiment of the present disclosure will be described in detail with reference to the accompanying configuration views or process flowcharts.

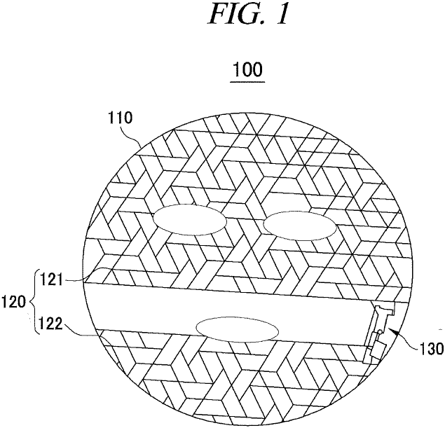

FIG. 1 illustrates a sheet mask device according to the present disclosure. Referring to FIG. 1, a sheet mask device 100 may include a base sheet 110, an electrode unit 120 and a power supply unit 130. The electrode unit 120 may include a first electrode pattern 121 and a second electrode pattern 122.

The sheet mask device 100 according to an embodiment of the present disclosure generates a microcurrent when a cosmetic composition permeates into the sheet mask and helps deliver the cosmetic composition deep into a skin.

Further, the sheet mask device 100 according to an embodiment of the present disclosure can exercise iontophoresis or galvanic functions on the skin through the electrode unit 120 and the power supply unit 130 formed on the base sheet 110 just by attaching the sheet mask onto the skin surface.

The base sheet 110 according to an embodiment of the present disclosure may have a shape to cover the skin. For example, the base sheet 110 may be a face-shaped sheet with holes for eyes and mouth, respectively.

The electrode unit 120 according to an embodiment of the present disclosure may be formed on the base sheet 110 to allow a current for iontophoresis to flow. For example, the electrode unit 120 changes an electrical environment of the skin by generating a potential difference on the skin and thus can increase the permeation of a liquid composition into the skin.

Referring to FIG. 1, the electrode unit 120 may include the first electrode pattern 121 and the second electrode pattern 122. For example, the first electrode pattern 121 and the second electrode pattern 122 may regularly form a pattern of a predetermined shape which is not limited to the embodiment shown in FIG. 1.

The first electrode pattern 121 may be formed on a part of the base sheet 110 and connected to an electrode of a battery 131. For example, the first electrode pattern 121 may be formed on an upper part of the base sheet 110 and may include a regularly connected pattern of a predetermined shape.’

The second electrode pattern 122 may be formed on another part of the base sheet 110 and spaced apart from the first electrode pattern 121, and may be connected to another electrode of the battery 131. For example, the second electrode pattern 122 may be formed on a lower part of the base sheet 110 and may include a repeatedly connected pattern of a predetermined shape.

The base sheet 110 may allow a microcurrent to flow into the skin through the first electrode pattern 121 and the second electrode pattern 122.

The electrode unit 120 may be formed of a material containing silver (Ag) by sputtering on the base sheet 110. For example, the first electrode pattern 121 and the second electrode pattern 122 may be formed of a silver-containing material with high electric conductivity.

Herein, the sputtering as a kind of vacuum deposition method is a process in which a target material is bombarded by energetic ions of plasma generated at a relatively low vacuum level and atoms are ejected from the target material by bombardment with the target material and deposited on a substrate to form a layer of the sputtered material.

For example, the first electrode pattern 121 and the second electrode pattern 122 may be formed by sputtering silver on the base sheet 110.

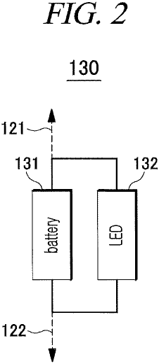

FIG. 2 is a conceptual diagram illustrating a power supply unit shown in FIG. 1. Referring to FIG. 2, the power supply unit 130 according to an embodiment of the present disclosure may be formed on the base sheet 110 and connected to the electrode unit 120 to supply electric energy. The power supply unit 130 may include the battery 131 and a display unit 132.

For example, the first electrode pattern 121 may be connected to one side of the battery 131, and the second electrode pattern 122 may be connected to the other side of the battery 131. Also, the battery 131 may be connected to the display unit 132.

For example, the power supply unit 130 may supple electric energy to each of the first electrode pattern 121 and the second electrode pattern 122. The display unit 132 may receive information about whether or not the supply of electric energy from the power supply unit 130 to the electrode unit 120 is activated.

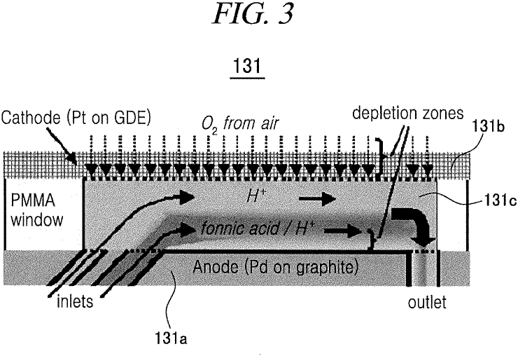

FIG. 3 is a conceptual diagram illustrating a battery shown in FIG. 2.

The battery 131 may be activated by a liquid composition supplied to the base sheet 110. For example, when the liquid composition for activating the battery 131 is supplied to the base sheet 110, the battery 131 may allow a current to flow to each of the first electrode pattern 121 and the second electrode pattern 122. For another example, when the liquid cosmetic composition is supplied to the base sheet 110, a current may be generated in each of the first electrode pattern 121 and the second electrode pattern 122, and, thus, a microcurrent may be allowed to flow into the skin.

Referring to FIG. 3, the battery 131 may include an anode 131a, a cathode 131b and an electrolyte layer 131c. The anode 131a may be configured to receive the liquid composition, the cathode 131b may be configured to receive an oxygen from an air, and the electrolyte layer 131c may be interposed between the anode 131a and the cathode 131b.

For example, when the liquid composition is supplied to the base sheet 110, the anode 131a receives the liquid composition and generates electrons through oxidation, whereas the cathode 131b consumes electrons through reduction.

For example, when the battery 131 is activated by the liquid composition supplied to the base sheet 110, positive ions are attracted toward the anode 131a that donates electrons and negative ions are attracted toward the cathode 131b that accepts electrons, and, thus, a current flows in the electrolyte layer 131c. The current may be allowed to flow to the first electrode pattern 121 and the second electrode pattern 122 connected to the battery 131.

For another example, when a liquid cosmetic composition is supplied to the base sheet 110, a current may be generated through oxidation and reduction occurring in the anode 131a, the cathode 131b and the electrolyte layer 131c and the generated current may be allowed to flow to the first electrode pattern 121 and the second electrode pattern 122.

The sheet mask device 100 according to an embodiment of the present disclosure can deliver the liquid cosmetic composition deep into the skin by allowing a microcurrent to flow into the skin through oxidation and reduction occurring in the battery 131.

That is, the sheet mask device 100 may activate the battery 131 with the liquid composition absorbed in the base sheet 110 to allow a current to flow to the electrode unit 120 including the first electrode pattern 121 and the second electrode pattern 122 and thus may have the same effect as iontophoresis therapy or a galvanic device just by attaching the sheet mask.

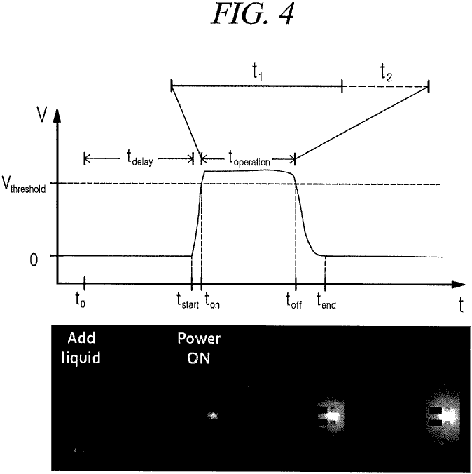

FIG. 4 shows an example operation of a display unit shown in FIG. 2. Referring to FIG. 4, the display unit 132 may visually display whether or not the battery 131 is activated. For example, when the liquid composition is absorbed in the base sheet 110, the display unit 132 may visually display the activation of the battery 131.

The display unit 132 may include an LED element that is turned on by electric energy supplied from the battery 131. For example, when a liquid cosmetic composition is supplied to the base sheet 110, the display unit 132 may display the activation of the battery 131 by turning on the LED element.

The LED element according to an embodiment of the present disclosure may be turned on for a predetermined amount of time from when the battery 131 is activated. For example, the LED element may be turned on for 10 minutes from when the liquid composition is absorbed in the base sheet 110 and the battery 131 is activated.

For another example, the LED element may be turned on from when a liquid cosmetic composition is absorbed in the base sheet 110 and the battery 131 is activated to when the liquid cosmetic composition is sufficiently dried. Specifically, referring to FIG. 4, there may be a delay time t_{delay} until the display unit 132 turns on light while a liquid composition is sufficiently absorbed in the base sheet 110. That is, there may be a delay time in turning on light by the display unit 132 until the battery 131 is fully activated.

After the liquid composition is sufficiently absorbed in the base sheet 110, i.e., when the battery 131 is fully activated, the display unit 132 turns on light for a predetermined amount of time t₁. Then, when the liquid composition absorbed in the base sheet 110 is dried for a sufficient amount of time, for example, when a voltage of the battery 131 is lowered to be equal to or less than a predetermined value or when a predetermined amount of time elapses, the display unit 132 may turn off light gradually for time t₂ or may turn on light for the time t₂ in a different pattern from that for the time t₁.

More specifically, the times t₁ and t₂ taken to turn on light by the display unit 132 may be determined by the amount of electric energy consumed.

For example, while oxidation and reduction are continued in the battery 131 by supplying a large amount of liquid composition to the base sheet 110, the times t₁ and t₂ taken to turn on light by the display unit 132 may increase. For another example, when a small amount of liquid composition is supplied to the base sheet 110, the times t₁ and t₂ taken to turn on light by the display unit 132 may relatively decrease. The times t₁ and t₂ taken to turn on light by the display unit 132 may also be determined by the amount of liquid composition supplied to the base sheet 110 which determines the amount of electric energy.

The LED element may change in at least one of a flicker pattern, brightness or color depending on an intensity of electric energy supplied from the battery 131 or the amount of time elapsed. For example, the intensity of electric energy supplied from the battery 131 may be divided into a plurality of levels, and a flicker pattern or brightness of the LED element of the display unit 132 may vary depending on the intensity of electric energy supplied. Specifically, a flicker rate or brightness of the LED element may be proportional to the intensity of electric energy supplied by the battery 131.

For another example, the intensity of electric energy supplied from the battery 131 may be divided into strong, medium and weak, and a color of the LED element of the display unit 132 may vary depending on the intensity of electric energy supplied.

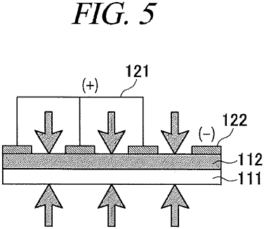

FIG. 5 is a cross-sectional view of a base sheet shown in FIG. 1.

The base sheet 110 according to an embodiment of the present disclosure may include a first sheet 111 and a second sheet 112. The first sheet 111 may be a fibrous sheet to be in contact with the skin, and the second sheet 112 may be formed of a metal-containing material on the first sheet 111 and may be coupled to the electrode unit 120.

For example, the base sheet 110 may have a dual structure including the first sheet 111 which is fibrous and the second sheet 112 formed of a metallic material. Specifically, the second sheet 112 formed of a metallic material is formed on a surface to be in contact with the skin, i.e., an outer surface of the first sheet 111 which is fibrous, and, thus, it is possible to inhibit evaporation of moisture while the sheet mask is used and also possible to supply a highly nutritional composition to the skin and improve the skin conditions.

For example, the first electrode pattern 121 and the second electrode pattern 122 may be formed on the second sheet 112 formed of a metallic material. Herein, the first electrode pattern 121 may be an anode, and the second electrode pattern 122 may be a cathode. When the battery 131 is activated, the electrode unit 120 may use a current supplied from the battery 131 to allow a microcurrent to flow into the skin through the second sheet 112.

Meanwhile, a case (not shown) according to an embodiment of the present disclosure may accommodate the base sheet 110 and the liquid composition to be isolated from each other, and may deisolate the base sheet 110 and the liquid composition when a user applies a pressure to the case.

For example, the boundary between the base sheet 110 and the liquid composition accommodated in the case to be isolated from each other may be opened when the user applies a predetermined pressure to the case. That is, the liquid composition is absorbed into the base sheet 110 by pressure of the user, and, thus, the battery 131 can be activated. The display unit 132 may visually display the activation of the battery 131 by turning on the LED element or the like.

The above description of the present disclosure is provided for the purpose of illustration, and it would be understood by a person with ordinary skill in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

We claim:

1. A sheet mask device that is attached to a skin and delivers a liquid composition into the skin, comprising:
a base sheet having a shape to cover the skin;
an electrode unit formed on the base sheet to allow a current for iontophoresis to flow; and
a power supply unit formed on the base sheet and connected to the electrode unit to supply electric energy,
wherein the power supply unit includes:
a battery activated by the liquid composition supplied to the base sheet; and
a display unit configured to visually display whether or not the battery is activated.

2. The sheet mask device of claim 1,
wherein the display unit includes an LED element that is turned on by electric energy supplied from the battery.

3. The sheet mask device of claim 2,
wherein the LED element changes in at least one of a flicker pattern, brightness or color depending on an intensity of the electric energy supplied from the battery.

4. The sheet mask device of claim 2,
wherein the LED element is turned on for a predetermined amount of time from when the battery is activated.

5. The sheet mask device of claim 1, further comprising:
a case that accommodates the base sheet and the liquid composition to be isolated from each other, and deisolates the base sheet and the liquid composition when a user applies a pressure to the case.

6. The sheet mask device of claim 1,
wherein the battery includes:
an anode configured to receive the liquid composition;
a cathode configured to receive an oxygen from an air; and
an electrolyte layer interposed between the anode and the cathode.

7. The sheet mask device of claim 1,
wherein the electrode unit includes:
a first electrode pattern formed on a part of the base sheet and connected to an electrode of the battery; and
a second electrode pattern formed on another part of the base sheet and spaced apart from the first electrode pattern, and connected to the other electrode of the battery.

8. The sheet mask device of claim 1,
wherein the electrode unit is formed of a material containing silver (Ag) by sputtering on the base sheet.

9. The sheet mask device of claim 7,
wherein the base sheet includes:
a first sheet that is a fibrous sheet to be in contact with the skin; and
a second sheet that is formed of a metal-containing material on the first sheet and is coupled to the electrode unit.
